(19) United States
(12) Patent Application Publication
(10) Pub. No.: US 2009/0010868 A1
(43) Pub. Date: Jan. 8, 2009

(54) COMPOSITION COMBINING A SILICONE POLYMER AND A TACKIFYING RESIN

(75) Inventors: Philippe ILEKTI, Maison-Alfort (FR); Laure Le Chaux, Chevilly La Rue (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/164,181

(22) Filed: Jun. 30, 2008

Related U.S. Application Data
(60) Provisional application No. 60/929,897, filed on Jul. 17, 2007.

(30) Foreign Application Priority Data
Jul. 3, 2007 (FR) 07 56248

(51) Int. Cl. A61K 8/72; A61Q 19/00

(52) U.S. Cl. 424/78.02

(57) ABSTRACT

The present invention relates to a solid composition containing, in a physiologically acceptable medium, at least one fatty phase containing:
at least one resin with a number-average molecular weight of less than or equal to 10 000 g/mol, chosen from rosin, rosin derivatives and hydrocarbon-based resins, and
at least one silicone polyamide,
   the silicone polyamide containing at least one silicone portion with a mean degree of polymerization of greater than or equal to 50 and representing at least 10% of the total weight of the composition.

### REFERENCE TO PRIOR APPLICATIONS

This application claims priority to U.S. provisional application 60/929,897 filed Jul. 17, 2007, and to French patent application 0756248 filed Jul. 3, 2007, both incorporated herein by reference.

### FIELD OF THE INVENTION

The present invention relates to a solid composition, especially to a cosmetic composition such as a makeup and/or care composition that may be applied to human skin, lips or integuments, for instance the hair, the eyelashes, the eyebrows or the nails, comprising at least one liquid fatty phase comprising at least one silicone polyamide and at least one tackifying resin.

In a preferred embodiment the present invention relates to a solid cosmetic composition comprising, in a physiologically acceptable medium, at least one fatty phase comprising:
at least one resin with a number-average molecular weight of less than or equal to 10 000 g/mol, chosen from rosin, rosin derivatives and hydrocarbon-based resins, and mixtures thereof, and
at least one silicone polyamide,
   the silicone polyamide comprising at least one silicone portion with a mean degree of polymerization of greater than or equal to 50 and representing at least 10% of the total weight of the composition.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is common to find a structured, i.e. gelled and/or rigidified, liquid phase in cosmetic or dermatological products. This is especially the case in solid compositions, in particular solid cast compositions, lip balms and lipsticks, eyeshadows, concealer products and cast foundations. This structuring is conventionally obtained with the aid of waxes or fillers or, more recently, using specific gelling agents.

Thus, documents WO-A-97/36573, U.S. Pat. No. 5,874,069, U.S. Pat. No. 5,919,441, U.S. Pat. No. 6,051,216, WO-A-02/17870, WO-A-02/17871, EP-A-1 177 784, WO-A-99/06473 and U.S. Pat. No. 6,353,076, which is a division of U.S. Pat. No. 6,051,216, and also document EP 1 695 693 propose cosmetic compositions such as deodorant sticks or gels, comprising a silicone oily phase gelled with a silicone polymer of polysiloxane/polyamide type.

The use of these silicone polymers allows access to a cosmetic composition with a novel solid texture, i.e. having relatively low rigidity and high elasticity. This texture does not correspond either to that of a conventional stick endowed with relatively high rigidity, or to that of a standard gel whose consistency is liquid or pasty.

### OBJECTS OF THE INVENTION

In conjunction with texture adjustment, the inventors sought to obtain a composition formulation that also has good deformability qualities for easy and pleasant application, and that is also, if need be, satisfactory in terms of gloss. In addition, the inventors sought to obtain a composition that shows good stability, and in particular to limit the phenomenon of exudation, especially in warm environments.

### SUMMARY OF THE INVENTION

The inventors observed, unexpectedly, that the combination of at least one silicone polyamide and of a tackifying resin makes it possible to obtain a composition that shows improved resistance on application, and in particular provides a composition that avoids becoming fractured during use. The inventors have thus found, unexpectedly, that the use of a tackifying resin in combination with a silicone polyamide produces compositions that are satisfactory in these terms.

### DESCRIPTION OF THE PREFERRED EMBODIMENTS

Consequently, according to a first aspect, the present invention relates to a composition, preferably a solid makeup or care or cosmetic composition, comprising, in a physiologically acceptable medium, at least one fatty phase comprising:
at least one resin with a number-average molecular weight of less than or equal to 10 000 g/mol, chosen from rosin, rosin derivatives, and hydrocarbon-based resins, and mixtures thereof, and
at least one silicone polyamide,
   the silicone polyamide comprising a silicone portion with a mean degree of polymerization of greater than or equal to 50 and representing at least 10% of the total weight of the composition.

The term “solid” characterizes the state of the composition at room temperature (25° C.) and at atmospheric pressure (760 mmHg).

More preferably, the silicone polyamide represents at least 15%, and better still at least 20%, of the total weight of the composition, including 25, 30, 35, 40, 50, etc. % and all values and subranges between stated values.

Preferably, the composition has an elasticity of greater than 80% including 85, 90, 100, etc. % and all values and subranges between stated values. Specifically, the combination of the resin and of at least one silicone polyamide for which the mean degree of polymerization of the silicone portion is greater than or equal to 50 and representing at least 10%, or better still 15%, or better still 20% by weight the composition makes it possible to obtain a composition with an elasticity of greater than 80% and in particular greater than 90%.

The invention allows the production of compositions with improved glidance that are pleasant to apply. The deposit obtained is uniform and shows improved gloss.

A subject of the present invention is also a process for making up or caring for keratin materials and especially the lips, in which a composition as defined above is applied to the lips.

Similarly, according to another of its aspects, the invention relates to the use of a silicone polyamide in combination with the resin, to prepare a stable cosmetic composition that can afford a film of improved gloss.

Unexpectedly, the solid compositions according to the invention thus prove to be endowed with an elastic texture, which may be associated with a particular application action.

For example, the supple, elastic solid textures obtained according to the invention may be compatible with direct application, for example to the lips, without requiring the use of an applicator as in the case of fluid textures. The compositions according to the invention also display, during application, suppleness, softness and very good elasticity, thus preserving the product for a future application. It is possible for the product not to be permanently deformed, and for it to regain its initial form after application.

In the form of a dome, the compositions in accordance with the invention advantageously display the behaviour of a deformable, supple elastic solid, which imparts noteworthy softness on application.

Furthermore, the compositions according to the invention have improved glidance and disintegration on application, which is therefore more pleasant.

### Characterization of the Elasticity and the Hardness

Advantageously, the compositions according to the invention have a hardness ranging from 10 to 250 g including 20, 40, 60, 80, 100, 120, 140, 160, 180, 200, 220, and 240 g, including all values and subranges between stated values, and/or an elasticity of greater than 80%.

The hardness and the elasticity of the composition according to the invention may be measured using a texturometer, which makes it possible to obtain the variation of the resistance to deformation of the composition as a function of the displacement of a spindle into a sample of the composition.

The texturometer measures the force of resistance to deformation of the composition once the spindle comes into contact with the sample. After having reached a maximum programmed depth L0 into the sample, the spindle returns to the initial point.

The hardness (expressed in grams or in newtons) is equal to the resistance value of the composition when the spindle is at the end of its course, and the elasticity (expressed as a percentage) is equal to the ratio of i) the distance L at which contact is broken between the spindle and the sample during the withdrawal of the spindle, and ii) the distance L0. The breaking of contact is reflected by the disappearance of the force of resistance of the composition on the spindle.

The elasticity is proportional to the distance over which the system “accompanies” the return of the spindle; the higher the value, the more elastic the system.

The texturometer used preferably is or is equivalent to a Stable Micro System TAX-T2i® texturometer equipped with exploitation software such as Texture Expert Exceed® equipped with a hemispherical P/0.5 HS Rheo plastic spindle.

The parameters applied are, or are equivalent to, the following:
speed before contact: 0.1 mm·s⁻¹,
speed of displacement into the sample: 0.1 mm·s⁻¹,
withdrawal speed: 0.1 mm·s⁻¹,
maximum depth L0:1 mm.

The composition samples are prepared by hot-casting a sufficient amount of the test composition, for example into a pretared 100×15 mm Petri dish, to obtain a sample about 1 cm thick. The advantage of the choice of this conditioning is its width that is sufficient to overcome any edge effects. Two Petri dishes are thus prepared, and are left to stand for a minimum of 24 hours at 20° C. before characterization.

At least three measurements are taken on each sample (Petri dish): one at the centre, and two others at points equidistant from the centre and from the edge of the dish.

The hardness and the elasticity are equal to the mean of the measurements taken.

The hardness of the composition according to the invention is such that the composition is self-supporting and can disintegrate easily to form a satisfactory deposit on the skin and the lips. In addition, with this hardness, the composition of the invention shows good impact strength.

More particularly, the hardness may range from 30 g to 200 g, especially from 50 g to 190 g, or even from 70 g to 175 g and more particularly from 100 g to 150 g.

The compositions according to the invention advantageously have an elasticity of greater than 90%.

### Characterization of the Stability

Samples of the composition are prepared by hot-casting (at about 100° C.) a sufficient amount of the test composition, for example into a 100×15 mm Petri dish, to obtain a sample about 1 cm thick. The Petri dish is left to stand for a minimum of 24 hours at 20° C. The samples are then stored at room temperature, on the one hand, and at 47° C., on the other hand, for two months.

The exudation is reflected by the appearance of oil droplets at the surface of the sample.

This test allows the stability of the composition to be evaluated at room temperature and at 47° C.

### Silicone Polyamide

As indicated previously, the compositions according to the invention comprise at least one silicone polyamide.

The silicone polyamides of the composition are preferably solid at room temperature (25° C.) and atmospheric pressure (760 mmHg).

For the purposes of the invention, the term “polymer” means a compound containing at least 2 repeating units, preferably at least 3 repeating units and better still 10 repeating units.

The silicone polyamides of the composition of the invention may be polymers of the polyorganosiloxane type, for instance those described in documents U.S. Pat. No. 5,874,069, U.S. Pat. No. 5,919,441, U.S. Pat. No. 6,051,216 and U.S. Pat. No. 5,981,680, all incorporated herein by reference. According to the invention, the silicone polymers may belong to the following two families:
(1) polyorganosiloxanes comprising at least two amide groups, these two groups being located in the polymer chain, and/or
(2) polyorganosiloxanes comprising at least two amide groups, these two groups being located on grafts or branches.

A) According to a first variant, the silicone polymers are polyorganosiloxanes as defined above in which the amide units are located in the polymer chain.

The silicone polyamides may be more particularly polymers comprising at least one unit corresponding to the general formula I:

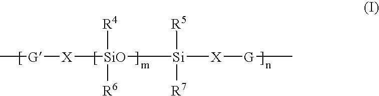

in which:
1) G′ represents C(O) when G represents —C(O)—NH—Y—NH—, and G′ represents —NH— when G represents —NH—C(O)—Y—C(O)—,
2) R⁴, R⁵, R⁶ and R⁷, which may be identical or different, represent a group chosen from:
   linear, branched or cyclic, saturated or unsaturated, C₁-C₄₀ hydrocarbon-based groups, possibly containing in their chain one or more oxygen, sulfur and/or nitrogen atoms, and possibly being partially or totally substituted with fluorine atoms,
   C₆-C₁₀ aryl groups, optionally substituted with one or more C₁-C₄ alkyl groups,
   polyorganosiloxane chains possibly containing one or more oxygen, sulfur and/or nitrogen atoms;
3) the groups X, which may be identical or different, represent a linear or branched C₁-C₃₀ alkylenediyl group, possibly containing in its chain one or more oxygen and/or nitrogen atoms;
4) Y is a saturated or unsaturated, C₁-C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃-C₈ cycloalkyl, C₁-C₄₀ alkyl, C₅-C₁₀ aryl, phenyl optionally substituted with 1 to 3 C₁-C₃ alkyl, C₁-C₃ hydroxyalkyl and C₁-C₆ aminoalkyl groups; or
5) Y represents a group corresponding to the formula:

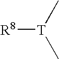

in which
   T represents a linear or branched, saturated or unsaturated, C₃-C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
   R⁸ represents a linear or branched C₁-C₅₀ alkyl group or a polyorganosiloxane chain, possibly comprising one or more ester, amide, urethane, thiocarbamate, urea, thiourea and/or sulfonamide groups, which may possibly be linked to another chain of the polymer;
6) n is an integer ranging from 2 to 500 and preferably from 2 to 200, and m is an integer ranging from 50 to 1000, preferably from 50 to 700 and better still from 50 to 200.

It will be noted that “m” corresponds to the mean degree of polymerization of the silicone portion of the silicone polyamide.

According to the invention, 80% of the groups R⁴, R⁵, R⁶ and R⁷ of the polymer are preferably chosen from methyl, ethyl, phenyl and 3,3,3-trifluoropropyl groups. According to another embodiment, 80% of the groups R⁴, R⁵, R⁶ and R⁷ of the polymer are methyl groups.

According to the invention, Y can represent various divalent groups, furthermore optionally comprising one or two free valencies to establish bonds with other units of the polymer or copolymer. Preferably, Y represents a group chosen from:
a) linear C₁ to C₂₀ and preferably C₁ to C₁₀ alkylene groups,
b) C₃₀ to C₅₆ branched alkylene groups possibly comprising rings and unconjugated unsaturations,
c) C₅-C₆ cycloalkylene groups,
d) phenylene groups optionally substituted with one or more C₁ to C₄₀ alkyl groups,
e) C₁ to C₂₀ alkylene groups comprising from 1 to 5 amide groups,
f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,
g) polyorganosiloxane chains of formula:

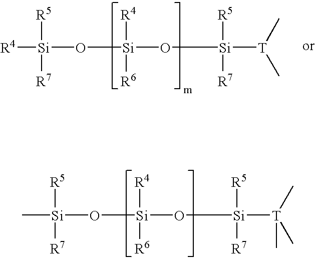

in which R⁴, R⁵, R⁶, R⁷, T and m are as defined above.

B) According to the second variant, the silicone polyamides may be polymers comprising at least one unit corresponding to formula (II):

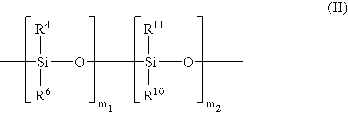

in which
R⁴ and R⁶, which may be identical or different, are as defined above for formula (I),
R¹⁰ represents a group as defined above for R⁴ and R⁶, or represents a group of formula —X-G″—R¹² in which X is as defined above for formula (I) and R¹² represents a hydrogen atom or a linear, branched or cyclic, saturated or unsaturated, C₁-C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁-C₄ alkyl groups,
and G″ represents —C(O)NH— and —HN—C(O)—,
R¹¹ represents a group of formula —X-G″—R¹² in which X, G″ and R¹² are as defined above,
m₁ is an integer ranging from 1 to 998, and
m₂ is an integer ranging from 2 to 500.

It will be noted that “m₁” corresponds to the mean degree of polymerization of the silicone portion of the silicone polyamide.

According to the invention, the silicone polymer may be a homopolymer, that is to say a polymer comprising several identical units, in particular units of formula (I) or of formula (II).

According to the invention, it is also possible to use a polymer that is a copolymer comprising several different units of formula (I), that is to say a polymer in which at least one of the groups R⁴, R⁵, R⁶, R⁷, X, G, Y, m and n is different in one of the units. The copolymer may also be formed from several units of formula (II), in which at least one of the groups R⁴, R⁶, R¹⁰, R¹¹, m₁ and m₂ is different in at least one of the units.

It is also possible to use a polymer comprising at least one unit of formula (I) and at least one unit of formula (II), the units of formula (I) and the units of formula (II) possibly being identical to or different from each other.

According to one variant of the invention, it is also possible to use a polymer furthermore comprising at least one hydrocarbon-based unit comprising two amide groups, chosen from ester, amide, sulfonamide, carbamate, thiocarbamate, urea, urethane, thiourea, oxamido, guanidino and biguanidino groups, and combinations thereof.

These copolymers may be block polymers or grafted polymers.

In formulae (I) and (II), the alkylene group representing X or Y can optionally contain in its alkylene part at least one of the following components:
1) one to five amide, urea, urethane or carbamate groups,
2) a C₅ or C₆ cycloalkyl group, and
3) a phenylene group optionally substituted with 1 to 3 identical or different C₁-C₃ alkyl groups.

In formulae (I) and (II), the alkylene groups may also be substituted with at least one component chosen from the group consisting of:
a hydroxyl group,
a C₃-C₈ cycloalkyl group,
one to three C₁-C₄₀ alkyl groups,
a phenyl group optionally substituted with one to three C₁-C₃ alkyl groups,
a C₁-C₃ hydroxyalkyl group, and
a C₁-C₆ aminoalkyl group.

In these formulae (I) and (II), Y may also represent:

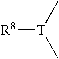

in which R⁸ represents a polyorganosiloxane chain and T represents a group of formula:

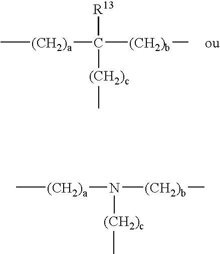

in which a, b and c are, independently, integers ranging from 1 to 10, and R¹³ is a hydrogen atom or a group such as those defined for R⁴, R⁵, R⁶ and R⁷.

In formulae (I) and (II), R⁴, R⁵, R⁶ and R⁷ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different units of formula (I) or (II).

Thus, the polymer may be a polyamide containing several units of formula (I) or (II) of different lengths, i.e. a polyamide corresponding to formula (III):

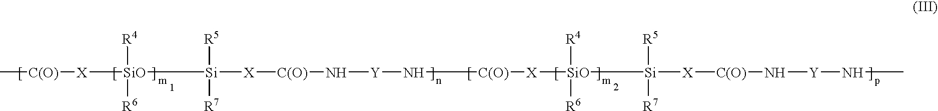

in which X, Y, n and R⁴ to R⁷ have the meanings given above, m₁ and m₂, which are different, are chosen in the range from 1 to 1000, and p is an integer ranging from 2 to 300.

In this formula, the units may be structured to form either a block copolymer, or a random copolymer or an alternating copolymer. In this copolymer, the units may be not only of different lengths, but also of different chemical structures, for example containing different groups Y. In this case, the polymer may correspond to formula IV:

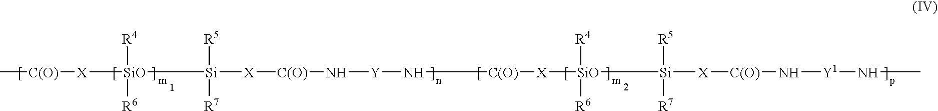

in which R⁴ to R⁷, X, Y, m₁, m₂, n and p have the meanings given above and Y¹ is different from Y but chosen from the groups defined for Y. As previously, the various units may be structured to form either a block copolymer, or a random copolymer or an alternating copolymer.

In this first embodiment of the invention, the silicone polymer may also consist of a grafted copolymer. Thus, the polyamide containing silicone units may be grafted and optionally crosslinked with silicone chains containing amide groups. Such polymers may be synthesized with trifunctional amines.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to one embodiment of the invention, a copolymer of silicone polyamide and of hydrocarbon-based polyamide, or a copolymer comprising units of formula (I) or (II) and hydrocarbon-based polyamide units, may be used. In this case, the polyamide-silicone units may be located at the ends of the hydrocarbon-based polyamide.

Advantageously, the composition comprises at least one polyamide/polydimethylsiloxane polymer, especially a polymer of general formula (I) with an index m of greater than 50, in particular greater than 75 and especially of about 100.

Advantageously, the silicone polyamide of formula (I) has a weight-average molecular mass ranging from 10 000 to 500 000 g/mol.

More preferably, X and Y independently represent a group chosen from linear C₁-C₂₀ and preferably C₁-C₁₀ alkylene groups.

As examples of polymers that may be used, mention may be made of one of the silicone polyamides obtained in accordance with Examples 1 to 3 of document U.S. Pat. No. 5,981,680, such as the product sold under the reference DC 2-8179 by Dow Corning.

According to one embodiment variant of the invention, the polymer consists of a homopolymer or copolymer comprising urethane or urea groups. These polymers are described in detail in patent application WO 2003/106 614.

The first composition may contain, in place of the silicone polyamide, a polyorganosiloxane polymer containing two or more urethane and/or urea groups, either in the backbone of the polymer or on side chains or as pendent groups.

The polymers comprising at least two urethane and/or urea groups in the backbone may be polymers comprising at least one unit corresponding to the following formula:

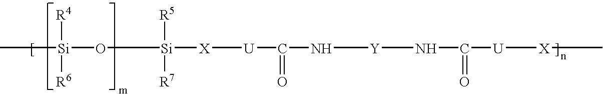

in which R⁴, R⁵, R⁶, R⁷, X, Y, m and n have the meanings given above for formula (I), and U represents —O— or —NH—, such that:

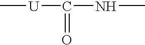

corresponds to a urethane or urea group.

In this formula, Y may be a linear or branched C₁-C₄₀ alkylene group, optionally substituted with a C₁-C₁₅ alkyl group or a C₅-C₁₀ aryl group. Preferably, a —(CH₂)₆— group is used.

The polymer constituting the silicone polymer may be formed from silicone-urethane and/or silicone-urea units of different length and/or constitution, and may be in the form of block, sequenced or statistical (random) copolymers.

As in the case of the silicone polyamides of formula (I), (II) or (III), silicone polyurethanes or polyureas having units of different length and structure, in particular units of different lengths via the number of silicone units, may be used in the invention.

The polymers and copolymers used in the composition of the invention advantageously have a transition temperature from the solid state to the liquid state ranging from 45° C. to 190° C. Preferably, they have a transition temperature from the solid state to the liquid state ranging from 70 to 130° C. and better still from 80° C. to 105° C.

The silicone polyamide may be present in the first composition in a total content ranging from 10% to 70% by weight relative to the total weight of the composition, preferably ranging from 10% to 50% by weight, better still ranging from 15% to 30% by weight and preferably ranging from 20% to 30% by weight relative to the total weight of the composition.

According to one advantageous embodiment, the composition comprises at least two silicone polyamides, in particular two silicone polyamides each independently comprising at least one unit of formula (I). The silicone polyamides may have, for example, a silicone portion with a different mean degree of polymerization.

Preferably, according to this embodiment, the composition comprises:
at least one first silicone polyamide comprising at least one unit of formula (I) as defined above in which m ranges from 50 to 600, in particular from 60 to 400 and especially from 75 to 200, and is more particularly about 120 for the first polymer, and
at least one second silicone polyamide comprising at least one second unit of formula (I) as defined above in which G′, G, R⁴, R⁵, R⁶, R⁷, X, Y and n are as defined above and m ranges from 5 to 100, in particular from 10 to 75, and is more particularly about 15 for the second polymer.

Advantageously, the first polymer has a weight-average molecular mass of between 10 000 and 500 000 g/mol. Advantageously, the second polymer preferably has a weight-average molecular mass ranging from 1000 to 500 000 g/mol and especially between 10 000 and 300 000 g/mol.

### Resin

The resin used in the composition according to the invention preferably has a number-average molecular weight of less than or equal to 10 000 g/mol, especially ranging from 250 to 10 000 g/mol, preferably less than or equal to 5000 g/mol, especially ranging from 250 to 5000 g/mol, better still less than or equal to 2000 g/mol, especially ranging from 250 to 2000 g/mol and better still less than or equal to 1000 g/mol, especially ranging from 250 to 1000 g/mol.

The number-average molecular weights (Mn) are determined by gel permeation liquid chromatography (THF solvent, calibration curve established with linear polystyrene standards, refractometric detector).

The resin of the composition according to the invention is advantageously a tackifying resin. Such resins are described especially in the Handbook of Pressure Sensitive Adhesive, edited by Donatas Satas, 3rd edition, 1989, pp. 609-619, incorporated herein by reference.

The resin of the composition according to the invention may preferably be chosen from rosin, rosin derivatives and hydrocarbon-based resins, and mixtures thereof.

Rosin is a mixture predominantly comprising organic acids known as rosin acids (mainly acids of abietic type and of pimaric type).

At least three types of rosin exist: rosin (“gum rosin”) obtained by incision on live trees, wood rosin, which is extracted from pine wood or stumps, and tall oil (“tall oil rosin”), which is obtained from a by-product originating from the production of paper.

The rosin derivatives may be derived in particular from the polymerization, hydrogenation and/or esterification (for example with polyhydric alcohols such as ethylene glycol, glycerol or pentaerythritol) of rosin acids. Examples that may be mentioned include the rosin esters sold under the reference Foral 85, Pentalyn H and Staybelite Ester 10 by the company Hercules; Sylvatac 95 and Zonester 85 by the company Arizona Chemical, or Unirez 3013 by the company Union Camp.

The hydrocarbon-based resins are chosen from low molecular weight polymers that may be classified, according to the type of monomer they comprise, as:
indene hydrocarbon-based resins such as the resins derived from the polymerization in major proportion of indene monomer and in minor proportion of monomers chosen from styrene, methylindene and methylstyrene, and mixtures thereof, these resins possibly being hydrogenated. These resins may have a molecular weight ranging from 290 to 1150 g/mol.
Examples of indene resins that may be mentioned include those sold under the reference Escorez 7105 by the company Exxon Chem., Nevchem 100 and Nevex 100 by the company Neville Chem., Norsolene S105 by the company Sartomer, Picco 6100 by the company Hercules and Resinall by the company Resinall Corp., or the hydrogenated indene/methylstyrene/styrene copolymers sold under the name “Regalite” by the company Eastman Chemical, in particular Regalite R1100, Regalite R1090, Regalite R7100, Regalite R1010 Hydrocarbon Resin and Regalite R1125 Hydrocarbon Resin;
aliphatic pentanediene resins such as those derived from the majority polymerization of the 1,3-pentanediene (trans or cis-piperylene) monomer and of minor monomers chosen from isoprene, butene, 2-methyl-2-butene, pentene and 1,4-pentanediene, and mixtures thereof. These resins may have a molecular weight ranging from 1000 to 2500 g/mol.
Such 1,3-pentanediene resins are sold, for example, under the references Piccotac 95 by the company Eastman Chemical, Escorez 1304 by the company Exxon Chemicals, Nevtac 100 by the company Neville Chem. or Wingtack 95 by the company Goodyear;
mixed resins of pentanediene and of indene, which are derived from the polymerization of a mixture of pentanediene and indene monomers such as those described above, for instance the resins sold under the reference Escorez 2101 by the company Exxon Chemicals, Nevpene 9500 by the company Neville Chem., Hercotac 1148 by the company Hercules, Norsolene A 100 by the company Sartomer, and Wingtack 86, Wingtack Extra and Wingtack Plus by the company Goodyear;
diene resins of cyclopentadiene dimers such as those derived from the polymerization of first monomers chosen from indene and styrene, and of second monomers chosen from cyclopentadiene dimers such as dicyclopentadiene, methyldicyclopentadiene and other pentanediene dimers, and mixtures thereof. These resins generally have a molecular weight ranging from 500 to 800 g/mol, for instance those sold under the reference Betaprene BR 100 by the company Arizona Chemical Co., Neville LX-685-125 and Neville LX-1000 by the company Neville Chem., Piccodiene 2215 by the company Hercules, Petro-Rez 200 by the company Lawter or Resinall 760 by the company Resinall Corp.;
diene resins of isoprene dimers such as terpenic resins derived from the polymerization of at least one monomer chosen from α-pinene, β-pinene and limonene, and mixtures thereof. These resins may have a molecular weight ranging from 300 to 2000 g/mol. Such resins are sold, for example, under the names Piccolyte A115 and S125 by the company Hercules, and Zonarez 7100 or Zonatac 105 Lite by the company Arizona Chem.

Mention may also be made of certain modified resins such as hydrogenated resins, for instance those sold under the name Eastotac C6-C20 Polyolefin by the company Eastman Chemical Co., under the reference Escorez 5300 by the company Exxon Chemicals, or the resins Nevillac Hard or Nevroz sold by the company Neville Chem., the resins Piccofyn A-100, Piccotex 100 or Piccovar AP25 sold by the company Hercules or the resin SP-553 sold by the company Schenectady Chemical Co.

According to one preferred embodiment, the resin is chosen from indene hydrocarbon-based resins, in particular the hydrogenated indene/methylstyrene/styrene copolymers sold under the name “Regalite” by the company Eastman Chemical, such as Regalite R1100, Regalite R1090, Regalite R7100, Regalite R1010 Hydrocarbon Resin and Regalite R1125 Hydrocarbon Resin.

The resin may be present in the composition according to the invention in a content ranging from 0.1% to 30% by weight, preferably from 0.3% to 20% by weight and better still from 0.5% to 15% by weight relative to the total weight of the composition, including all values and subranges between stated values.

Preferably, the tackifying resin is present in the composition in a ratio such that the silicone polyamide/resin mass proportion is greater than 2 and better still ranges from 2 to 40, including 4, 6, 8, 10, 15, 20, 25, 20, 25 and 38, and all values and subranges between stated values.

### Liquid Fatty Phase

The fatty phase of the composition according to the invention is preferably a liquid fatty phase based on at least one oil. The fatty phase will sometimes be described as a “(liquid) fatty phase”, meaning it can be a liquid or a non-liquid phase, the term including both.

The oil may be a silicone oil, an ester oil or a non-silicone oil.

a. Silicone Oil

According to one variant of the invention, the (liquid) fatty phase comprises at least one silicone oil. This oil may be a volatile oil or a non-volatile oil.

For the purposes of the invention, a volatile oil has at room temperature (25° C.) and atmospheric pressure (760 mmHg) a vapour pressure ranging from 0.02 mmHg to 300 mmHg (2.66 Pa to 40 000 Pa) and better still ranging from 0.1 mmHg to 90 mmHg (13 Pa to 12 000 Pa). The non-volatile oils then correspond to a vapour pressure of less than 0.02 mmHg (2.66 Pa) and better still less than 10⁻³ mmHg (0.13 Pa).

The volatile silicone oil may be chosen from linear or cyclic silicone oils, such as linear or cyclic polydimethylsiloxanes (PDMS) containing from 3 to 7 silicon atoms.

Examples of such oils that may be mentioned include octyl trimethicone, hexyl trimethicone, decamethylcyclopentasiloxane (cyclopentasiloxane or D5), octamethylcyclotetrasiloxane, (cyclotetradimethylsiloxane or D4), dodecamethylcyclohexasiloxane (D6), decamethyltetrasiloxane (L4), KF 96 A from Shin-Etsu, and polydimethylsiloxanes such as those sold under the references DC 200 (1.5 cSt), DC 200 (5 cSt) and DC 200 (3 cSt) from Dow Corning.

The non-volatile silicone oils may be polydimethylsiloxanes, polyalkylmethylsiloxanes, dimethicone copolyols, alkylmethicone copolyols, cetyl dimethicone, silicones containing alkylglyceryl ether groups, silicones containing amine side groups and dilauroyltrimethylolpropane siloxysilicate. The alkyl groups of these oils especially contain from 2 to 24 carbon atoms.

The non-volatile silicone oils that may be used in the liquid fatty phase may in particular be linear non-volatile polydimethylsiloxanes (PDMS) that are liquid at room temperature; polydimethylsiloxanes comprising alkyl, alkoxy or phenyl groups, which are pendent and/or at the end of a silicone chain, these groups each containing from 2 to 24 carbon atoms; fluoro silicones with groups that are pendent or at the end of a chain, containing from 1 to 12 carbon atoms, all or some of the hydrogen atoms of which are substituted with fluorine atoms, dimethiconols, and mixtures thereof.

According to one preferred embodiment, the composition according to the invention comprises a phenyl silicone as non-volatile silicone oil. The term “phenyl silicone” means an organopolysiloxane substituted with at least one phenyl group.

The phenyl silicone is preferably non-volatile.

Preferably, the molecular weight of the phenyl silicone oil is between 500 and 10 000 g/mol.

The silicone oil may be chosen from phenyl trimethicones, phenyl dimethicones, phenyltrimethylsiloxydiphenylsiloxanes, diphenyl dimethicones and diphenylmethyldiphenyltrisiloxanes.

The silicone oil may correspond to the formula:

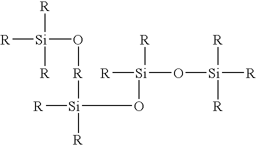

in which the groups R represent, independently of each other, a methyl or a phenyl. Preferably, in this formula, the silicone oil comprises at least three, for example at least four, at least five or at least six phenyl groups.

According to another embodiment, the silicone oil corresponds to the formula

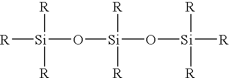

in which the groups R represent, independently of each other, a methyl or a phenyl. Preferably, in this formula, the organopolysiloxane comprises at least three, for example at least four or at least five phenyl groups.

Mixtures of silicone oils may be used. Thus, mixtures of the phenyl organopolysiloxanes described above may be used.

Examples that may be mentioned include mixtures of triphenyl, tetraphenyl or pentaphenyl organopolysiloxane.

According to another embodiment, the silicone oil corresponds to the formula

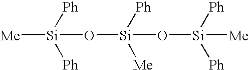

in which Me represents methyl and Ph represents phenyl. Such a phenyl silicone is especially manufactured by Dow Corning under the reference Dow Corning 555 Cosmetic Fluid: trimethyl pentaphenyl trisiloxane. The reference Dow Corning 554 Cosmetic Fluid may also be used.

The silicone oils have a viscosity advantageously chosen in the range from 5 to 800 000 cSt, preferably from 10 to 500 000 cSt and better still from 10 to 5000 cSt at 25° C.

The (liquid) fatty phase advantageously contains from 0.1% to 70%, for example from 5% to 60%, by weight of silicone oil(s).

Preferably, the silicone oil(s) represent(s) at least 20% and better still at least 30% relative to the total weight of the composition.

b. Ester Oil

According to one variant of the invention, at least one of the oils of the (liquid) fatty phase is an oil known as an “ester oil”, which is chosen from esters of monocarboxylic acids with monoalcohols and polyalcohols.

Advantageously, the ester corresponds to the following formula:

R₁—CO—O—R₂

where R₁ represents a linear or branched alkyl radical of 1 to 40 carbon atoms and preferably of 7 to 19 carbon atoms, optionally comprising one or more ethylenic double bonds, and optionally substituted.

R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

Preferably, the total number of carbon atoms of R₁+R₂ is ≧9.

R₁ may represent a linear or, preferably, branched fatty acid residue containing from 1 to 40 and even better from 7 to 19 carbon atoms, and R₂ may represent a linear or, preferably, branched hydrocarbon-based chain containing from 1 to 40, preferably from 3 to 30 and even better from 3 to 20 carbon atoms. Once again, preferably the number of carbon atoms of R₁+R₂≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters that may be used in the fatty phases of the compositions of the invention include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

Advantageously, the esters are chosen from the compounds of formula (I) above, in which R₁ represents an unsubstituted linear or branched alkyl group of 1 to 40 carbon atoms and preferably of 7 to 19 carbon atoms, optionally comprising one or more ethylenic double bonds, and R₂ represents an unsubstituted linear or branched alkyl group of 1 to 40 carbon atoms, preferably of 3 to 30 carbon atoms and even better of 3 to 20 carbon atoms, optionally comprising one or more ethylenic double bonds.

Preferably, R₁ is an unsubstituted branched alkyl group of 4 to 14 carbon atoms and preferably of 8 to 10 carbon atoms, and R₂ is an unsubstituted branched alkyl group of 5 to 15 carbon atoms and preferably of 9 to 11 carbon atoms. Preferably, in formula (I), R₁—CO— and R₂ have the same number of carbon atoms and are derived from the same radical, preferably an unsubstituted branched alkyl, for example isononyl, i.e. the ester oil molecule is advantageously symmetrical.

The ester oil will preferably be chosen from the following compounds:
isononyl isononanoate,
cetostearyl octanoate,
isopropyl myristate,
2-ethylhexyl palmitate,
2-octyldodecyl stearate,
2-octyldodecyl erucate,
isostearyl isostearate.

The ester that is preferred among all of them is isononyl isononanoate.

According to one embodiment, the composition comprises less than 10% of volatile ester oil, preferably less than 5% of volatile ester oil and better still less than 3% of volatile ester oil, or is even free of volatile ester oil.

Advantageously, the (liquid) fatty phase comprises from 0.1% to 60% by weight and preferably from 5% to 50% by weight of ester oil(s).

c. Non-Silicone Oil

The (liquid) fatty phase of the compositions according to the invention may also contain one or more volatile or non-volatile non-silicone oils. The volatile non-silicone oils may be chosen from the group of volatile hydrocarbon-based oils, esters and ethers, such as volatile hydrocarbons, for instance isododecane and isohexadecane, and C₈-C₁₆ isoparaffins.

The term “hydrocarbon-based oil” means an oil mainly containing hydrogen and carbon atoms, and optionally oxygen, nitrogen, sulfur and/or phosphorus atoms.

The volatile non-silicone oil may also be chosen from fluoro oils such as perfluoropolyethers, perfluoroalkanes, for instance perfluorodecalin, perfluoroadamantanes, perfluoroalkyl phosphate monoesters, diesters and triesters, and fluoro ester oils.

As examples of volatile non-silicone oils that may be used in the composition of the invention, mention may be made of isododecane, isohexadecane, propylene glycol n-butyl ether, ethyl 3-ethoxypropionate, propylene glycol methyl ether acetate, C₁₁-C₁₃ isoparaffins such as Isopar L® or C₁₁-C₁₂ isoparaffins such as Isopar H®.

When the fatty phase comprises a volatile non-silicone oil, it advantageously represents from 0.1% to 60% and better still from 5% to 20%, relative to the total weight of the composition.

The (liquid) fatty phase may also contain other non-silicone oils, for example polar oils such as:
hydrocarbon-based plant oils with a high content of triglycerides consisting of fatty acid esters of glycerol in which the fatty acids may have varied chain lengths, these chains possibly being linear or branched, and saturated or unsaturated; these oils are in particular wheatgerm oil, corn oil, sunflower oil, shea oil, castor oil, sweet almond oil, macadamia oil, apricot oil, soybean oil, rapeseed oil, cottonseed oil, alfalfa oil, poppy seed oil, pumpkin seed oil, sesame seed oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passion flower oil and musk rose oil; or caprylic/capric acid triglycerides such as those sold by the company Stearines Dubois or those sold under the names Miglyol 810, 812 and 818 by the company Dynamit Nobel;
synthetic ethers containing from 10 to 40 carbon atoms;
C₈ to C₂₆ fatty alcohols, for instance oleyl alcohol and octyldodecanol;
fatty acids, for instance oleic acid, linoleic acid or linolenic acid; and
mixtures thereof.

The liquid fatty phase may also contain apolar oils such as linear or branched, volatile or non-volatile hydrocarbons or fluorocarbons of synthetic or mineral origin, for instance liquid paraffins (such as isoparaffins) and derivatives thereof, petroleum jelly, polydecenes, hydrogenated polyisobutene such as Parleam, and squalane, and mixtures thereof.

The oil(s) (volatile or non-volatile) may be present in the composition according to the invention in a content ranging from 0.1% to 90% by weight and preferably from 5% to 80% by weight relative to the total weight of the composition.

In one particular embodiment, the composition comprises less than 10%, or less than 5%, of volatile oil relative to the total weight of the composition, or is even free of volatile oil.

### Active Agents

The composition according to the invention may also comprise at least one active agent. The term “active agent” means a compound that has a cosmetic and/or dermatological effect especially on the lips.

This active agent may be hydrophilic or hydrophobic. The active agent may be water-soluble.

Thus, the active agent present in the composition according to the invention may be chosen independently from:
dermo-relaxing agents,
agents for stimulating the synthesis of dermal or epidermal macromolecules and/or for preventing their degradation,
anti-glycation agents,
anti-irritants,
moisturizers,
desquamating agents,
pigmentation modifiers,
NO-synthase inhibitors,
agents for stimulating fibroblast or keratinocyte proliferation and/or keratinocyte differentiation,
anti-pollution agents or free-radical scavengers,
calmatives,
agents acting on the capillary circulation,
agents acting on the energy metabolism of cells,
cicatrizing agents, and
mixtures thereof.

The amount of active agent(s) ranges, for example, from 0.0001% to 30% by weight and preferably from 0.01% to 20% by weight of active material relative to the total weight of the composition.

### Physiologically Acceptable Medium

The composition of the invention preferably is cosmetically or dermatologically acceptable, i.e. it contains a non-toxic physiologically acceptable medium that may be applied to, e.g., human lips. For the purposes of the invention, the term “cosmetically acceptable” refers to a composition of pleasant appearance, odour and feel.

### Structuring Agent

The composition according to the invention may comprise, besides the silicone polyamide(s) described above, a structuring agent chosen from waxes, semi-crystalline polymers and lipophilic gelling agents, and mixtures thereof.

It is understood that the amount of these additional compounds may be adjusted by a person skilled in the art so as not to harm the desired effect in the context of the present invention.

### Wax(es)

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

In particular, the waxes that are suitable for the invention may have a melting point of greater than or equal to 45° C. and in particular greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:
A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the invention are chosen from waxes that are solid at room temperature of animal, plant, mineral or synthetic origin, and mixtures thereof.

As illustrations of waxes that are suitable for the invention, mention may be made especially of hydrocarbon-based waxes, for instance beeswax, lanolin wax, Chinese insect waxes, rice bran wax, carnauba wax, candelilla wax, ouricurry wax, esparto grass wax, berry wax, shellac wax, Japan wax and sumach wax; montan wax, orange wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes (C₃₀₋₄₅ alkyl dimethicone) and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

A wax that may be used is a C₂₀-C₄₀ alkyl (hydroxystearyloxy)stearate (the alkyl group containing from 20 to 40 carbon atoms), alone or as a mixture.

Such a wax is especially sold under the names Kester Wax K 82 P®, Hydroxypolyester K 82 P® and Kester Wax K 80 P® by the company Koster Keunen.

As microwaxes that may be used in the compositions according to the invention, mention may be made especially of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The composition according to the invention may comprise a content of waxes ranging from 0.1% to 30% by weight relative to the total weight of the composition; it may in particular contain from 0.5% to 15% and more particularly from 1% to 10% thereof.

According to another embodiment, the composition according to the invention is free of wax.

### Pasty Compounds

The composition according to the invention may also contain a pasty compound, which may be chosen advantageously from:
lanolin and its derivatives
polymeric or non-polymeric silicone compounds
polymeric or non-polymeric fluoro compounds
vinyl polymers, especially:
   olefin homopolymers
   olefin copolymers
   hydrogenated diene homopolymers and copolymers
   linear or branched oligomers, homopolymers or copolymers of alkyl (meth)acrylates preferably containing a C₈-C₃₀ alkyl group
   oligomers, homopolymers and copolymers of vinyl esters containing C₈-C₃₀ alkyl groups
   oligomers, homopolymers and copolymers of vinyl ethers containing C₈-C₃₀ alkyl groups
liposoluble polyethers resulting from the polyetherification between one or more C₂-C₁₀₀ and preferably C₂-C₅₀ diols
esters, and
mixtures thereof.

Among the esters, the following are especially preferred:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, especially such as those sold under the brand name Softisan 649 by the company Sasol,
the arachidyl propionate sold under the brand name Waxenol 801 by Alzo,
phytosterol esters,
fatty acid triglycerides and derivatives thereof,
pentaerythritol esters,
non-crosslinked polyesters resulting from the polycondensation between a linear or branched C₄-C₅₀ dicarboxylic acid or polycarboxylic acid and a C₂-C₅₀ diol or polyol,
aliphatic esters of an ester, resulting from the esterification of an aliphatic hydroxycarboxylic acid ester with an aliphatic carboxylic acid,
polyesters resulting from the esterification, with a polycarboxylic acid, of an aliphatic hydroxycarboxylic acid ester, the ester comprising at least two hydroxyl groups, such as the products Risocast DA-H® and Risocast DA-L®, and
mixtures thereof.

Among the pasty compounds of plant origin that will preferably be chosen is a mixture of oxyethylenated (5 OE) oxypropylenated (5 OP) soybean sterols and pentaerythritol, sold under the reference Lanolide by the company Vevy.

According to one embodiment, the composition comprises less than 10% by weight, preferably less than 7%, better still less than 5% and even better still less than 3% by weight of pasty fatty substance relative to the total weight of the composition. More preferably, the composition is totally free of pasty fatty substance.

### Silicone Resin

The composition according to the invention advantageously contains at least one silicone resin.

Examples of these silicone resins that may be mentioned include:
siloxysilicates, which may be trimethylsiloxysilicates of formula [(CH₃)₃SiO]ₓ(SiO_{4/2})_{y} (units MQ) in which x and y are integers ranging from 50 to 80,
polysilsesquioxanes of formula (CH₃SiO_{3/2})ₓ (units T) in which x is greater than 100 and at least one of the methyl radicals of which may be substituted with a group R as defined above,
polymethylsilsesquioxanes, which are polysilsesquioxanes in which none of the methyl radicals is substituted with another group. Such polymethylsilsesquioxanes are described in document U.S. Pat. No. 5,246,694, the content of which is incorporated by reference.

As examples of commercially available polymethylsilsesquioxane resins, mention may be made of those sold:
by the company Wacker under the reference Resin MK, such as Belsil PMS MK: polymer comprising CH₃SiO_{3/2} repeating units (units T), which may also comprise up to 1% by weight of (CH₃)₂SiO_{2/2} units (units D) and having an average molecular weight of about 10 000,
by the company Shin-Etsu under the references KR-220L, which are composed of units T of formula CH₃SiO_{3/2} and contain Si—OH (silanol) end groups, under the reference KR-242A, which comprise 98% of units T and 2% of dimethyl units D and contain Si—OH end groups, or under the reference KR-251, comprising 88% of units T and 12% of dimethyl units D and contain Si—OH end groups.

Siloxysilicate resins that may be mentioned include trimethylsiloxysilicate resins (TMS) optionally in the form of powders. Such resins are sold under the reference SR1000 by the company General Electric or under the reference TMS 803 by the company Wacker. Mention may also be made of trimethylsiloxysilicate resins sold in a solvent such as cyclomethicone, sold under the name KF-7312J by the company Shin-Etsu or DC 749 and DC 593 by the company Dow Corning.

Advantageously, the silicone resin, for instance the trimethyl siloxysilicate resin, is present in a content ranging from 0.5% to 30%, or better still from 1% to 25% or even better still from 5% to 25% relative to the total weight of the composition.

Preferably, the silicone resin, and especially the trimethyl siloxysilicate resin, is present in a ratio such that the silicone polyamide/silicone resin mass proportion is between 1/4 and 4 and preferably between 1/3 and 3.

### Lipophilic Gelling Agents

The gelling agents that may be used in the compositions according to the invention may be organic or mineral, polymeric or molecular lipophilic gelling agents.

Mineral lipophilic gelling agents that may be mentioned include optionally modified clays, for instance hectorites modified with a C₁₀-C₂₂ ammonium chloride, for instance hectorite modified with distearyldimethylammonium chloride, for instance the product sold under the name Bentone 38V® by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:
trimethylsiloxyl groups, which are obtained especially by treating fumed silica in the presence of hexamethyldisilazane. Silicas thus treated are known as Silica Silylate according to the CTFA (8th edition, 2000). They are sold, for example, under the references Aerosil R812® by the company Degussa, and Cab-O-Sil TS-530® by the company Cabot;
dimethylsilyloxyl or polydimethylsiloxane groups, which are obtained especially by treating fumed silica in the presence of polydimethylsiloxane or dimethyldichlorosilane. Silicas thus treated are known as Silica Dimethyl Silylate according to the CTFA (8th edition, 2000). They are sold, for example, under the references Aerosil R972® and Aerosil R974® by the company Degussa, and Cab-O-Sil TS-610® and Cab-O-Sil TS-720® by the company Cabot.

The hydrophobic fumed silica preferably has a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF10®, SR-DC556®, SR 5CYC Gel®, SR DMF 10 Gel® and SR DC 556 Gel® from Grant Industries and SF 1204® and JK 113® from General Electric; ethylcellulose, for instance the product sold under the name Ethocel® by Dow Chemical; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof. Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylenepropylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that may be used in the compositions according to the invention, mention may also be made of fatty acid esters of dextrin, such as dextrin palmitates, especially the products sold under the name Rheopearl TL® or Rheopearl KL® by the company Chiba Flour.

### Aqueous Phase

According to a first embodiment of the invention, the composition according to the invention may comprise an aqueous medium, constituting an aqueous phase, which may form the dispersed phase of the composition.

The aqueous phase may consist essentially of water; it may also comprise a mixture of water and of water-miscible solvent (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous phase (water and optionally the water-miscible solvent) may be present in a content ranging from 1% to 95% by weight, preferably ranging from 2% to 80% by weight and preferentially ranging from 3% to 60% by weight relative to the total weight of the composition.

The composition according to the invention may also contain less than 10% by weight or even less than 4% by weight of aqueous phase or of water, or even is totally anhydrous.

### Dyestuffs

According to one embodiment, the composition according to the invention may also comprise at least one colouring agent, which may be chosen from water-soluble or liposoluble dyes, pigments and nacres, and mixtures thereof.

The composition according to the invention may also comprise one or more dyestuffs chosen from water-soluble dyes and pulverulent dyestuffs, for instance pigments, nacres and glitter flakes that are well known to those skilled in the art. The dyestuffs may be present in the composition in a content ranging from 0.01% to 50% by weight and preferably from 0.01% to 30% by weight relative to the weight of the composition.

The term “pigments” should be understood as meaning white or coloured, mineral or organic particles, which are insoluble in an aqueous solution and which are intended to colour and/or opacify the resulting film.

The pigments may be present in a proportion of from 0.01% to 20% by weight, especially from 0.01% to 15% by weight and in particular from 0.02% to 10% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

They may also be pigments with a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment with a structure that may be, for example, of silica microsphere type containing iron oxide. An example of a pigment having this structure is the product sold by the company Miyoshi under the reference PC Ball® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542 669, EP-A-787 730, EP-A-787 731 and WO-A-96/08537.

The term “nacres” should be understood as meaning iridescent or non-iridescent coloured particles of any form, especially produced by certain molluscs in their shell, or else synthesized, and which have a colour effect by optical interference.

The nacres may be chosen from nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the nacres available on the market, mention may be made of the mica-based nacres Timica®, Flamenco® and Duochrome® sold by the company Engelhard, the Timiron® nacres sold by the company Merck, the Prestige® mica-based nacres, sold by the company Eckart, and the Sunshine® synthetic mica-based nacres, sold by the company Sun Chemical.

The nacres may more particularly have a yellow, pink, red, bronze, orangey, brown, gold and/or coppery colour or tint.

As illustrations of nacres that may be used in the context of the present invention, mention may be made especially of the gold-coloured nacres sold especially by the company Engelhard under the name Brillant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

The term “dyes” should be understood as meaning compounds that are generally organic, which are soluble in fatty substances such as oils or in an aqueous-alcoholic phase.

The cosmetic composition according to the invention may also comprise water-soluble or liposoluble dyes. The liposoluble dyes are, for example, Sudan red, DC Red 17, DC Green 6, β-carotene, Sudan brown, DC Yellow 11, DC Violet 2, DC Orange 5 and quinoline yellow. The water-soluble dyes are, for example, beetroot juice or methylene blue.

The cosmetic composition according to the invention may also contain at least one material with a specific optical effect.

This effect is different from a simple conventional hue effect, i.e. a unified and stabilized effect as produced by standard dyestuffs, for instance monochromatic pigments. For the purposes of the invention, the term “stabilized” means lacking an effect of variability of the colour as a function of the angle of observation or alternatively in response to a temperature change.

For example, this material may be chosen from particles with a metallic tint, goniochromatic colouring agents, diffracting pigments, thermochromic agents, optical brighteners, and also fibres, especially interference fibres. Needless to say, these various materials may be combined so as to simultaneously afford two effects, or even a novel effect in accordance with the invention.

The particles with a metallic tint that may be used in the invention are chosen in particular from:
particles of at least one metal and/or of at least one metal derivative,
particles comprising a mono-material or multi-material organic or mineral substrate, at least partially coated with at least one coat with a metallic tint comprising at least one metal and/or at least one metal derivative, and
mixtures of the particles.

Among the metals that may be present in the particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr and mixtures or alloys thereof (for example bronzes and brasses) are preferred metals.

The term “metal derivatives” is intended to denote compounds derived from metals, especially oxides, fluorides, chlorides and sulfides.

As illustrations of these particles, mention may be made of aluminium particles, such as those sold under the names Starbrite 1200 EAC® by the company Siberline, and Metalure® by the company Eckart.

Mention may also be made of copper metal powders or alloy mixtures such as the reference 2844 sold by the company Radium Bronze, metallic pigments such as aluminium or bronze, such as those sold under the name Rotosafe® 700 from the company Eckart, the silica-coated aluminium particles sold under the name Visionaire Bright Silver® from the company Eckart and metal alloy particles, for instance the silica-coated bronze (alloy of copper and zinc) powders sold under the name Visionaire Bright Natural Gold® from the company Eckart.

They may also be particles comprising a glass substrate, such as those sold by the company Nippon Sheet Glass under the name Microglass Metashine®.

The goniochromatic colouring agent may be chosen, for example, from multilayer interference structures and liquid-crystal colouring agents.

Examples of symmetrical multilayer interference structures that may be used in the compositions prepared in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair by the company Flex; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of the Infinite Colors® pigments from the company Shiseido. Depending on the thickness and the nature of the various layers, different effects are obtained. Thus, with the Fe₂O₃/SiO₂/Al/SiO₂/Fe₂O₃ structure, the colour changes from green-golden to red-grey for SiO₂ layers of 320 to 350 nm; from red to golden for SiO₂ layers of 380 to 400 nm; from violet to green for SiO₂ layers of 410 to 420 nm; from copper to red for SiO₂ layers of 430 to 440 nm.

Examples of pigments with a polymeric multilayer structure that may be mentioned include those sold by the company 3M under the name Color Glitter.

Examples of liquid-crystal goniochromatic particles that may be used include those sold by the company Chenix and also the products sold under the name Helicone® HC by the company Wacker.

### Filler

The composition according to the invention may comprise at least one filler, especially in a content ranging from 0.01% to 50% by weight and preferably ranging from 0.01% to 30% by weight relative to the total weight of the composition. The term “fillers” should be understood as meaning white or colourless, mineral or synthetic particles of any form, which are insoluble in the medium of the composition irrespective of the temperature at which the composition is manufactured. These fillers serve especially to modify the rheology or the texture of the composition.

The fillers may be mineral or organic of any form, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, expanded hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

### Additional Ingredients

The composition according to the invention may also comprise any cosmetic ingredient, which may be chosen especially from antioxidants, fragrances, preserving agents, neutralizers, surfactants, sunscreens, vitamins, moisturizers, self-tanning compounds, anti-wrinkle active agents, emollients, hydrophilic or lipophilic active agents, free-radical scavengers, deodorants, sequestrants and film-forming agents, and mixtures thereof.

Needless to say, a person skilled in the art will take care to select the optional additional ingredients and/or the amount thereof such that the advantageous properties of the composition according to the invention are not, or are not substantially, adversely affected by the envisaged addition.

The product according to the invention may be advantageously used for making up the skin and/or the lips and/or the integuments depending on the nature of the ingredients used. In particular, the product of the invention may be in the form of a solid foundation, a lipstick wand or paste, a concealer product, an eye contour product, an eyeliner, a mascara, an eyeshadow, a body makeup product or a skin colouring product.

The compositions of the product of the invention may be obtained by heating the various constituents to a temperature corresponding to the melting point of the highest-melting ingredient(s), followed by pouring the molten mixture into a mould (dish or finger stall). They may also be obtained by extrusion as described in patent application EP-A-0 667 146.

In particular, the composition of the invention may be in the form of a coloured lip makeup product such as a lipstick, a lip gloss or a pencil, optionally having care or treating properties. It may be in the form of an anhydrous stick.

The composition according to the invention may be manufactured via the known processes generally used in cosmetics or dermatology. For example, it may be manufactured via the following process:
In a first stage, the fillers and pigments may be ground in part of the oily phase.
The rest of the liposoluble ingredients may then be mixed together at a temperature of about 100° C. The ground material or the predispersed active agents may then be added to the oily phase.
The optional hydrophilic active agents may then be dispersed using a mechanical blender.
Finally, the composition may be poured into a mould suitable to give it a dome shape, and the whole may be left to cool to room temperature.

According to another aspect, the invention also relates to a cosmetic assembly comprising:
i) a container delimiting a compartment, the container being closed by means of a closing member; and
ii) a compound in accordance with the invention placed inside the compartment.

The container may be in any suitable form. It may especially be in the form of a jar, a case, a dish or a box.

The closing member may be in the form of a removable stopper, a lid, a cover, a tear-off strip or a capsule, especially of the type comprising a body fixed to the container and a cap articulated on the body. It may also be in the form of a member that selectively closes the container, especially a pump or a valve, for instance a flap valve.

The examples that follow are given as non-limiting illustrations of the present invention. The percentages are weight percentages.

### EXAMPLES 1 AND 2 OF LIPSTICKS

The compositions of Examples 1 and 2 are obtained according to the following protocol.

In a first stage, the fillers and pigments are ground in part of the oily phase.

The rest of the liposoluble ingredients and the surfactants (cetyl PEG/PPG-10/1 dimethicone and trioleyl phosphate) are then mixed together at a temperature of about 100° C. The ground material or the predispersed active agents are then added to the oily phase.

The hydrophilic active agents are then dispersed using a Moritz blender.

Finally, the composition is poured into a mould to give it a dome shape and the whole is left to cool to room temperature.

| | Example 1 | Example 2 |
|---|---|---|
| Compound (US INCI Name) | (comparative) | (Invention) |
| Lauroyl lysine | 2 | 2 |
| Sodium saccharin | 0.02 | 0.02 |
| Brown, yellow iron oxides | 0.02 | 0.02 |
| (75/25) (CI: 77491 + 77492) | | |
| Titanium dioxide | 0.50 | 0.50 |
| Red 28 lake | 0.0040 | 0.0040 |
| Fragrance | 0.30 | 0.30 |
| Hydrogenated polyisobutene | 15.11 | 13.11 |
| Hydrogenated | — | 2 |
| styrene/methylstyrene/indene | | |
| copolymer | | |
| (Regalite R 1100) | | |
| Dimethicone (viscosity: | 12.66 | 12.66 |
| 10 cSt) | | |
| Phenyl trimethicone | 36.306 | 36.306 |
| (viscosity: 20 cSt - MW: | | |
| 372) | | |
| Cetyl PEG/PPG-10/1 | 1 | 1 |
| dimethicone (20/75/5 - | | |
| viscosity: 3000 cSt) | | |
| Nylon-611/dimethicone | 22.50 | 22.50 |
| copolymer (= polyamide/poly- | | |
| dimethylsiloxane DC 2-8179 | | |
| from Dow Corning) (m > 50) | | |
| Nylon-611/dimethicone | 1.08 | 1.08 |
| copolymer (and) PPG-3 | | |
| myristyl ether (= poly- | | |
| amide/polydimethylsiloxane | | |
| DC 2-8178 from DowCorning) | | |
| (m < 50) | | |
| Glycerol | 7.50 | 7.50 |
| Trioleyl phosphate | 1 | 1 |
| Total | 100 | 100 |

For Comparative Example 1, a composition having the following is obtained:
a hardness of 156 g, and
an elasticity of 93%.

For Example 2 according to the invention, a composition having the following is obtained:
a hardness of 164 g, and
an elasticity of 93%.

The composition according to the invention has improved hardness without, however, losing any elasticity relative to the comparative example. The composition according to the invention thus has improved resistance and has less risk of becoming fractured during use.

Furthermore, it is found that the composition according to the invention of Example 2 has improved application qualities and an improved makeup result relative to Comparative Example 1, especially in terms of gloss and glidance on application, while at the same time maintaining hardness and elasticity that are comparable or superior to those of Example 1.

Moreover, an improvement in the stability of the composition according to the invention relative to that of the comparative example, in particular in terms of non-exudation, is also observed.

The above written description of the invention provides a manner and process of making and using it such that any person skilled in this art is enabled to make and use the same, this enablement being provided in particular for the subject matter of the appended claims, which make up a part of the original description and including a solid composition comprising, in a physiologically acceptable medium, at least one fatty phase comprising:
at least one resin with a number-average molecular weight of less than or equal to 10 000 g/mol, chosen from rosin, rosin derivatives and hydrocarbon-based resins, and mixtures thereof, and
at least one silicone polyamide,
   the silicone polyamide comprising at least one silicone portion with a mean degree of polymerization of greater than or equal to 50 and representing at least 10% of the total weight of the composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A solid composition comprising, in a physiologically acceptable medium, at least one fatty phase comprising:
at least one resin with a number-average molecular weight of less than or equal to 10 000 g/mol, chosen from rosin, rosin derivatives and hydrocarbon-based resins, and
at least one silicone polyamide,
the silicone polyamide comprising at least one silicone portion with a mean degree of polymerization of greater than or equal to 50 and representing at least 10% of the total weight of the composition.

2. The composition according to claim 1, wherein the silicone polyamide represents at least 15% relative to the total weight of the composition.

3. The composition according to claim 1, comprising less than 10% of water.

4. The composition according to claim 1, having a total content of silicone polyamides representing from 10% to 70% of the total weight of the composition.

5. The composition according to claim 1, comprising at least one silicone polyamide that comprises at least one unit corresponding to the general formula I: 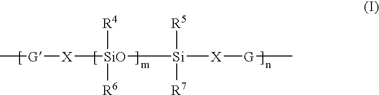
in which:
1) G′ represents C(O) when G represents —C(O)—NH—Y—NH—, and G′ represents —NH— when G represents —NH—C(O)—Y—C(O)—,
2) R⁴, R⁵, R⁶ and R⁷, which may be identical or different, represent a group chosen from:
linear, branched or cyclic, saturated or unsaturated, C₁-C₄₀ hydrocarbon-based groups, optionally containing in their chain one or more oxygen, sulfur and/or nitrogen atoms, and optionally being partially or totally substituted with fluorine atoms,
C₆-C₁₀ aryl groups, optionally substituted with one or more C₁-C₄ alkyl groups,
polyorganosiloxane chains optionally containing one or more oxygen, sulfur and/or nitrogen atoms;
3) the groups X, which may be identical or different, represent a linear or branched C₁-C₃₀ alkylenediyl group, optionally containing in its chain one or more oxygen and/or nitrogen atoms;
4) Y is a saturated or unsaturated, C₁-C₅₀ linear or branched divalent alkylene, arylene, cycloalkylene, alkylarylene or arylalkylene group, optionally comprising one or more oxygen, sulfur and/or nitrogen atoms, and/or bearing as substituent one of the following atoms or groups of atoms: fluorine, hydroxyl, C₃-C₈ cycloalkyl, C₁-C₄₀ alkyl, C₅-C₁₀ aryl, phenyl optionally substituted with 1 to 3 C₁-C₃ alkyl, C₁-C₃ hydroxyalkyl and C₁-C₆ aminoalkyl groups; or
5) Y represents a group corresponding to the formula: 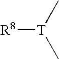
in which
T represents a linear or branched, saturated or unsaturated, C₃-C₂₄ trivalent or tetravalent hydrocarbon-based group optionally substituted with a polyorganosiloxane chain, and optionally containing one or more atoms chosen from O, N and S, or T represents a trivalent atom chosen from N, P and Al, and
R⁸ represents a linear or branched C₁-C₅₀ alkyl group or a polyorganosiloxane chain, optionally comprising one or more ester, amide, urethane, thiocarbamate, urea, thiourea and/or sulfonamide groups, which may optionally be linked to another chain of the polymer;
6) n is an integer ranging from 2 to 500, and m is an integer ranging from 50 to 1000.

6. The composition according to claim 5, in which X and/or Y represent(s) an alkylene group containing in its alkylene portion at least one of the following components:
1) 1 to 5 amide, urea, urethane or carbamate groups,
2) a C₅ or C₆ cycloalkyl group, and
3) a phenylene group optionally substituted with 1 to 3 identical or different C₁-C₃ alkyl groups, and/or substituted with at least one component chosen from the group consisting of:
a hydroxyl group,
a C₃-C₈ cycloalkyl group,
one to three C₁-C₄₀ alkyl groups,
a phenyl group optionally substituted with one to three C₁-C₃ alkyl groups,
a C₁-C₃ hydroxyalkyl group, and
a C₁-C₆ aminoalkyl group.

7. The composition according to claim 5, in which R⁴, R⁵, R⁶ and R⁷ independently represent a linear or branched C₁-C₄₀ alkyl group, a polyorganosiloxane chain or a phenyl group optionally substituted with one to three methyl or ethyl groups.

8. The composition according to claim 5, having an index m with a value of greater than 75.

9. The composition according to claim 5, comprising:
at least one first silicone polyamide comprising at least one unit of formula (I) in which m is 50 to 600, and
at least one second silicone polyamide comprising at least one unit of formula (I) in which m is 5 to 100 and is lower than the value of m in the first silicone polyamide.

10. The composition according to claim 1, comprising an indene hydrocarbon-based resin derived from the polymerization of an indene monomer and of a monomer chosen from styrene, methylindene and methylstyrene, and mixtures thereof.

11. The composition according to claim 1, comprising an indene hydrocarbon-based resin that is hydrogenated.

12. The composition according to claim 1, comprising an indene resin chosen from hydrogenated indene/methylstyrene/styrene copolymers.

13. The composition according to claim 1, comprising an aliphatic pentanediene resin derived from the polymerization of 1,3-pentanediene monomer (trans- or cis-piperylene) and of a monomer chosen from isoprene, butene, 2-methyl-2-butene, pentene and 1,4-pentanediene, and mixtures thereof.

14. The composition according to claim 1, comprising a mixed resin of pentanediene and of indene.

15. The composition according to claim 1, comprising a diene resin of cyclopentanediene dimers derived from the polymerization of a first monomer chosen from indene and styrene, and of a second monomer chosen from cyclopentanediene dimers.

16. The composition according to claim 1, comprising a diene resin of isoprene dimers derived from the polymerization of at least one monomer chosen from α-pinene, β-pinene and limonene, and mixtures thereof.

17. The composition according to claim 1, wherein the resin is present in a content ranging from 0.1% to 30% by weight relative to the total weight of the composition.

18. The composition according to claim 1, comprising a hydrogenated indene/methylstyrene/styrene copolymer and a Nylon-611/dimethicone copolymer.

19. The composition according to claim 1, further comprising at least one silicone oil representing from 0.1% to 70% relative to the total weight of the composition.

20. The composition according to claim 1, further comprising at least one silicone resin which is a siloxysilicate resin.

21. The composition according to claim 1, further comprising at least one pasty fatty substance.

22. The composition according to claim 1, further comprising at least one wax.

23. The composition according to claim 1, wherein it is free of wax.

24. The composition according to claim 1, further comprising at least one dyestuff.

25. A process comprising applying the composition according to claim 1 to the skin and/or the lips and/or the integuments.
